(19) United States
(12) Patent Application Publication
(10) Pub. No.: US 2005/0266455 A1
(43) Pub. Date: Dec. 1, 2005

(54) Method and microelectronic device for multi-site molecule detection

(75) Inventor: Valeri V. Golovlev, Oak Ridge, TN (US)

Correspondence Address: LUEDEKA, NEELY & GRAHAM, P.C., P O BOX 1871, KNOXVILLE, TN, 37901, US

(73) Assignee: SCI TEC, Inc.

(21) Appl. No.: 11/116,592

(22) Filed: Apr. 28, 2005

Related U.S. Application Data
(63) Continuation of application No. 10/086,805, filed on Mar. 2, 2002, now abandoned.

(51) Int. Cl. C12Q 1/68; C12M 1/34

(52) U.S. Cl. 435/6; 435/287.2

(57) ABSTRACT

A method and apparatus are provided for identifying molecular structures within a sample substance using an integrated circuit array sensor having a plurality of test sites upon which the sample substance is applied. Each test site includes a probe formed therein to bond with an associated target molecular structure. An electrical signal produced on the test site upon forming or breaking of molecular probe-target duplexes is detected to determine which probes have bonded to an associated target molecular structure.

### TECHNICAL FIELD OF THE INVENTION

The present invention relates to the field of bio-polymer analysis and detection which is of interest in biomedical research, genetic studies and disease diagnosis, toxicology tests, forensic investigation, and agriculture and pharmaceutical development

### BACKGROUND OF THE INVENTION

Nucleic acid hybridization has become an increasingly important technology for DNA analysis and gene expression studies. For example, DNA and RNA hybridization techniques are very useful for detecting, identifying, fingerprinting, and mapping molecular structures. Recently developed combinatorial DNA chips, which rely on the specific hybridization of target and probe DNA on a solid surface, attracted tremendous interest from the scientific and medical communities. A historical background as well as a description of the basic concept of bio-polymer arrays for the study and diagnostics of biological systems is provided in the following references:
A. M. Maxam and W. Gilbert, “A New Method for Sequencing DNA”, Proc. Natl. Acad. Sci. USA 74, 560-564 (1977)
Saiki et al, “Genetic Analysis of Amplified DNA with Immobilized Sequence-Specific Oligonucleotide Probes”, Proc. Natl. Acad Sci. USA 86, 6230-6234 (1989)
Chee et al, “Accessing Genetic Information With High-Density DNA Arrays”, Science 274, 5287 (1996)
Pastinen et al, “Minisequencing: A Specific Tool for DNA Analysis and Diagnostics on Oligonucleotide Arrays”, Genome Research 7, 606-614 (1997)
P. A. Fodor, “Techwire”, Science 277, 5324 (1998)
Landegren et al, “Reading Bits of Genetic Information: Methods for Single-Nucleotide Polymorphism Analysis”, Genome Research 8, 769-776 (1998)
Cho et al, “Parallel Analysis of Genetic Selections Using Whole Genome Oligonucleotide Arrays”, Proc. Natl. Acad. Sci. USA 95, 3752-3757 (1998)
Kricka et al, “Miniaturization of Analytical Sytems”, Clinical Chemistry 44:9, 2008-2014 (1998)
Southern et al, “Molecular Interactions on Microarrays”, Nature Genetics 21(1), 5-10 (1999)
Duggan et al, “Expression Profiling Using cDNA Microarrays”, Nature Genetics 21(1), 10-15 (1999)
Cheung et al, “Making and Reading Microarrays”, Nature Genetics 21(1), 15-20 (1999)
Lipshutz et al, “High Density Synthetic Oligonucleotide Arrays”, Nature Genetics 21(1), 20-25 (1999)
H. Ge, “UPA, a Universal Protein Array System For Quantitative Detection of Protein-Protein, Protein-DNA, Protein-RNA and Protein-Ligand Interactions”, Nucleic Acids Research 28(2), e3 (2000)
G. MacBeath, S. L. Schreiber, “Printing Proteins As Microarrays for High-Throughput Function Determination”, Science 289, 1760 (2000) see also
Hollis et al, (1998), U.S. Pat. No. 5,846,708
Wang et al, (1999), U.S. Pat. No. 5,922,617
Dale et al, (2000), U.S. Pat. No. 6,087,112;
Fodor (2001), U.S. Pat. No. 6,197,506;
Hori et al, (2001), U.S. Pat. No. 6,194,148;
Schwartz et al, (2001), U.S. Pat. No. 6,221,592
Fodor et al, (1992), Foreign Pat. No. WO92/10588
Virtanen, (1998), Foreign Pat. No. WO98/01533
Ribi, (1990), Foreign Pat. No. EP 0 402 917
   and references herein.

Although the study of gene activity and molecular mechanisms of disease and drug effects has traditionally focused on genomics, recently proteomics has introduced a very valuable complimentary approach to study the biological functions of a cell. Proteomics involves the qualitative and quantitative measurement of gene activity by detecting and quantifying expressions at the protein level, rather than at the messenger RNA level. Multianalyte assays, also known in the art as “protein chips”, involve the use of multiple antibodies and are directed towards assaying for multiple analytes. The approach enables rapid, simultaneous processing of thousands of proteins employing automation and miniaturization strategy introduced by DNA microarrays.

An attractive feature of microarray technology for genomic applications is that it has the potential to monitor the whole genome on a single chip, so that researchers can have a complete picture of the interaction among thousands of genes simultaneously. Possible applications of DNA microarrays include genetic studies and disease diagnosis, toxicology testing, forensic investigation, and agriculture and pharmaceutical development Growing applications for microarrays creates new demands for reducing the complexity and improving the detection sensitivity of DNA chips.

Currently, the most common approach to detect DNA bound to the microarray is to label it with a reporter molecule that identifies DNA presence. The reporter molecules emit detectable light when excited by an external light source. Light emitted by a reporter molecule has a characteristic wavelength, which is different from the wavelength of the excitation light, and therefore a detector such as a Charge-Coupled Device (CCD) or a confocal microscope can selectively detect a reporter's emission. Although the use of optical detection methods increases the throughput of the sequencing experiments, the disadvantages are serious. Incorporation of a fluorescent label into a nucleic acid sequence increases the complexity and cost of the entire process. Although the chemistry is commonplace, it necessitates an additional step. The increase in cost is due to the extra reagents necessary for fluorescent labeling, as well as precautionary steps necessary for safe handling of mutagenic materials.

Autoradiography is another common technique for detection of molecular structures. For DNA sequence analysis -applications, oligonucleotide fragments are end labeled, for example, with ³²P or ³⁵S. These end labeled fragments are then exposed to X-ray film for a specified amount of time. The amount of film exposure is determined by densitometry and is directly related to the amount of radioactivity of the labeled fragments adjacent to a region of film.

The use of any radioactive label is associated with several disadvantages. First, the use of radioactive isotopes increases the risk of workers acquiring mutation-related diseases. As such, precautions must be implemented when using radioactive markers or labels. Second, the need of an additional processing step and the use of additional chemical reagents and short-lived radioisotopes increases the cost and complexity of this detection technique.

The most relevant prior art to the present invention involves sensors that are based on electrical means for analyte detection. There are several classes of sensors that make use of applied electrical signals for determination of analyte presence. “Potentiometric” and “amperometric” sensors make use of oxidation-reduction chemistries in which electrons or electrochemically active species are generated or transferred due to analyte presence. An enzyme that interacts with an analyte may produce electrons that are delivered to an appropriate electrode; alternately an potentiometric sensor may employ two or more enzyme species, one interacting with the analyte, while the other actually generates electrons as a function of the action of the first enzyme (a “coupled” enzyme system). The general potentiometric method makes use of an applied voltage and the effects of electrochemically active species on said voltage. An example of a potentiometric sensor is described in Gaberlein et al, “Disposable potentiometric enzyme sensor for direct determination of organophosphorous insecticides”, Analyst, 125, 2274-2279 (2000), in which a organophosphorous sensor relies on electron transfer effected by a redox enzyme and electrochemically-active enzyme cofactor species. The present invention does not require application of an external voltage for pursuing oxidation/reduction chemistry, or electron generation/transfer.

An additional class of electrical sensing systems includes those sensors that make use primarily of changes in an electrical response of the sensor as a function of analyte presence, see, for example, U.S. Pat. No. 5,670,322 and U.S. Pat. No. 5,846,708 and references therein. A method for detecting molecular structures is taught by Eggers, et al, where a substance is applied to a plurality of test sites, each test site having a probe formed therein capable of binding to a known molecular structure. Electrical signals are collectively applied to the test sites, and electrical properties of the test sites are detected to determine whether the probe has bonded to an associated molecular structure. However, the need of applying an external electric field to testing sites also causes undesirable electrochemical processes that reduce the detection sensitivity and reproducibility of electrical sensing systems.

Other prior-art voltage-based sensors require the use of semiconducting field-effect transistors (FET's) and rely on the chemical generation or physical trapping of charged species near the sensor surface. This method has found widespread use in the detection of positively-charged heavy metals as well as analytes that are involved in proton (H⁺) generating enzyme reactions. Poghossian, et al, “Penicillin Detection by Means of Field-Effect Based Sensor: EnFET, EIS, or LAPS?”, Sensors and Actuators B78: 237-242 (2001), described a pH-sensitive enzymatic Field-Effect Transistor (EnFET) with immobilized β-lactamase. The pH-sensitive transducer detects variations in the H⁺-ion concentration resulting from the catalyzed hydrolysis of penicillin by the enzyme. The resulting local pH decrease near the pH-sensitive layer leads to a change in the drain current of the EnFED. However, FET-based biosensors known from the art generally suffer from a lack of sensitivity, low detection speed, and do not address the issue of integrating a large number of sensors for analysis of a plurality of target molecules in parallel. The present invention therefore provides an improved apparatus and method superior to that in the art.

While hundreds of sensors have been described in patents and in the scientific literature, actual commercial use of such sensors remains limited. In particular, virually all sensor designs set forth in prior art contain one or more inherent weaknesses. Some lack the sensitivity and/or speed of detection necessary to accomplish certain tasks. Other sensors lack long-term stability. Still others cannot be sufficiently miniaturized to be commercially viable or are prohibitively expensive to produce.

It is therefore a primary object of the present invention to provide an improved sensor, utilizing an array of miniaturized sensitive pixels. Said pixels are capable of detecting and outputting an electrical signal representative of the electric charge accumulated upon interaction of probe and target molecular structure.

It is yet another object of the invention to provide an improved method for detection and analysis of molecular structures by employing an integrated circuit array sensor having a plurality of test sites upon which the sample substance is applied, which said method is versatile in application, simple to use, and demonstrates the sensitivity and reproducibility necessary for commercial application.

Nomenclature

Unless defined otherwise, all technical and scientific terms used above and throughout the text have the same meaning as commonly understood to one of ordinary skill in the art to which this invention belongs.

The following definitions are provided to facilitate a clear understanding of the present invention. The term “molecular structure” refers to a macro-molecule, including organic compound, antibody, antigen, virus particle, metal complex, molecular ion, cellular metabolite, enzyme inhibitor, receptor ligand, nerve agent, peptide, protein, fatty acid, steroid, hormone, narcotic agent, synthetic molecule, medication, nucleic acid single-stranded or double-stranded polymer and equivalents thereof known in the art.

The term “bound molecular structures” or “duplex” refers to a corresponding pair of molecules held together due to mutual affinity or binding capacity, typically specific or non-specific binding or interaction, including biochemical, physiological, and/or pharmaceutical interactions. Herein binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “sample substance” refers to a media, often a liquid media, which was prepared for the purpose of analysis and establishing (a) the presence or absence of a particular type of molecular structure; (b) the presence or absence of a plurality of molecular structures; (c) the presence or absence of specific groups of molecular structures.

The term “target molecular structure” or “target” refers to a molecular structure whose presence or absence in a sample substance needs to be established.

The term “probe molecular structure” or “probe” refers to a molecular structure of known nature, which said probe is capable of binding to a particular type of target molecular structure or to any agent from a specific class of molecular structures. Said probe is used to witness the presence of the corresponding target molecular structure in a sample substance.

The terms “sensitive pixel” or “pixel unit” are used interchangeably and refer to a structural unit of the integrated circuit array sensor, herein said unit is designed to accumulate and convert an electric charge into an output electronic signal.

It must be noted that as used herein and in the appended claims, the singular forms “a”, “and”, and “the” include plural referents unless the context clearly dictates otherwise. Thus, for example, reference to “a molecular structure” may include a plurality of macro-molecules, including organic compounds, antibodies, antigens, virus particles, metals, metal complexes, ions, cellular metabolites, enzyme inhibitors, receptor ligands, nerve agents, peptides, proteins, fatty acids, steroids, hormones, narcotic agents, synthetic molecules, medications, nucleic acid single-stranded or double-stranded polymers and equivalents thereof known to those skilled in the art and so forth.

### SUMMARY OF THE INVENTION

The present invention provides an improved method and apparatus useful for detecting, identifying, fingerprinting, and mapping molecular structures. In accordance with the present invention, an apparatus and method capable of simultaneously detecting multiple molecular structures in predetermined test sites is provided. The method and apparatus provided herein substantially eliminates or prevents the disadvantages and problems associated with devices and methods known from prior art.

In the present invention identifying molecular structures within a sample substance is accomplished using an integrated circuit array sensor having a plurality of test sites upon which the sample substance is applied. Each test site includes a probe formed therein to bond with an associated target molecular structure. An electrical signal produced on the test site upon forming or breaking of molecular probe-target duplexes thereby is detected to determine which probes have bonded to an associated target molecular structure.

To eliminate or prevent the disadvantages and problems associated with devices and methods known from prior art, in the present invention no external electrical signals are applied collectively or separately to the test sites to determine electrical properties of said test sites. In the present invention, whether the probe has bonded to an associated molecular structure is determined by employing an integrated circuit array sensor capable of detecting an electrical signal produced on the test sites upon forming or melting of probe-target duplexes. To achieve the level of sensitivity required for reliable detection of the electrical signal produced on the test sites by probe-target duplexes, said integrated circuit array sensor comprises a set of miniaturized pixel units. Said integrated array sensor comprises at least one pixel unit, although said sensor often can comprise more than a million of the pixel units, and most preferably said sensor comprises more than a hundred thousand individual pixel units. Each said pixel unit is usually not bigger than (1 mm×1 mm) in size, and most preferably said pixel unit is less than (100 μm×100 μm) in size. Herein, reducing the size of the pixel contributes to a higher sensitivity of the sensor due to lower capacitive current, smaller ohmic drop and faster achievement of mass transport in a stationary diffusion state on the surface of sensitive elements of each individual pixel unit

In one embodiment of this invention, said integrated circuit array sensor comprises a set of active pixels, with each said pixel having one or more active transistors within the pixel unit, and multiple column readout circuits, similar to the architecture implemented by CMOS array imagers. Said active pixels are capable of converting the electrical charge accumulated by said pixel into an output electronic signal.

In yet another embodiment of this invention, said integrated circuit array sensor comprises an array of pixel units capable of accumulating, storing, and transferring an electrical charge to a readout register formed in the sensor's substrate, similar to the design implemented by a Charge-Coupled Device (CCD). Said pixels and readout circuit are capable of converting the electrical charge accumulated by said pixels into an output electronic signal.

A set of means known from prior art is provided to interface said integrated circuit array sensor with external control, post-processing, and data storage circuits.

To expose said integrated circuit array sensor to a sample substance, a chamber, such as a hybridization chamber, is installed or assembled on the sensitive area of said sensor.

Furthermore, the present invention discloses a method of identifying molecular structures within a sample substance, comprising the steps of:
(a) applying the substance to a plurality of test sites formed on a surface of said integrated circuit array sensor, said test sites having respective probes attached thereto which specifically bind to a target molecular structure, such that different test sites have probes which specifically bind to different target molecular structures; and such that each test site covers at least one pixel circuitry of said array sensor;
(b) maintaining a constant preprogrammed temperature of the substance and said integrated circuit array sensor, or alternatively, running a preprogrammed temperature profile such as to, but not limited to, gradually increase or decrease the temperature or effect a stepwise change of the temperature;
(c) acquiring an electronic signal from a plurality of the pixels associated with the test sites, each test site covering at least one pixel of said integrated array sensor,
(d) detecting the amplitude of the electronic signal versus time from the test sites to determine which probes have interacted with an associated target molecular structure such that a plurality of different targets can be detected.

Hereinabove, an innovative aspect of said method for identifying molecular structures comprises maintaining preprogrammed temperature profiles of the substance to which said integrated circuit array sensor is exposed. For example, target molecular structures can be identified based on specific values of temperature at which reactions, such as forming or breaking bonds, occur in response to a gradual increase or decrease of the temperature of the sample substance. Alternatively, target molecular structures can be identified based on the rate of forming or breaking bonds with molecular structures of the associated probe site in response to the stepwise change of temperature. Furthermore, the stepwise change of temperature herein provides the additional benefit of increasing the detection sensitivity by increasing the magnitude of electronic signals from probe sites by forcing the reaction of melting or breaking bonds occurring within a short time interval following the temperature rising above the melting point.

The present invention is distinguished from prior art in several ways. Firstly, the methodology is applicable to nearly all binding agents and not simply to those that produce or interact with electrons or electrochemically active compounds. Secondly, there is no requirement in the present invention for application of electromagnetic radiation, voltage, or electrical current Therefore, there is no undesirable electrochemical processes on the sensor's surface, which can modify test sites and affect reproducibility. Thirdly, the present invention provides enhanced detection sensitivity and time response because of employing miniaturized sensitive pixels with lower capacitive current and faster achievement of mass transport on the sensor's sensitive electrodes. In the following description, numerous specific details are set forth in order to provide a thorough understanding of the present invention. It will be apparent, however, to one skilled in the art that the present invention may be practiced without these specific details.

### BRIEF DESCRIPTION OF THE DRAWINGS

For a better understanding of these and other objectives of the present invention, reference is made to the following detailed description of the invention, by way of example, which is to be read in conjunction with the following drawings, wherein:

FIG. 1 illustrates a schematic representation of an electrode assembly of a single cell unit;

FIG. 2 illustrates a schematic representation of another embodiment of a single cell unit with the functionality of a metal-semiconductor diode detector;

FIG. 3 illustrates a schematic representation of a multi-electrode assembly;

FIG. 4 illustrates a schematic representation of an integrated array sensor;

FIG. 5 illustrates a 2×2 fragment of the array sensor with individually addressable active pixels;

FIG. 6 illustrates a 3×3 fragment of the array sensor implementing architecture of shift registers known from prior art;

FIG. 7 illustrates a particular example of apparatus for the detection of biomolecules in sample media using an integrated circuit array sensor;

FIG. 8 illustrates a 2D pattern of an electronic signal acquired by an integrated circuit array sensor having a 3×3 array of probe sites;

FIG. 9 illustrates a 2D pattern of an electronic signal versus time acquired from a single probe site upon binding probe and target molecular structures;

FIG. 10 illustrates a signal acquired from a single cell unit upon changing the temperature of the sample substance;

FIG. 11 illustrates signal versus temperature for (a) a perfect match and (b) a one base mismatch of probe-target oligos.

### DETAILED DESCRIPTION OF THE INVENTION

Hereinafter, embodiments for carrying out the present invention will be described in the case of using DNA samples and DNA probes. However, it would be obvious to those skilled in the art that embodiments herein are only illustrative of the present invention. It is also to be understood that the terminology used above and throughout the text is for the purpose of describing this particular embodiment only, and is not intended to limit the scope of the present invention that will be limited only by the appended claims.

All publications mentioned are incorporated herein by reference for the purpose of describing and disclosing, for example, materials, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### The Invention in General

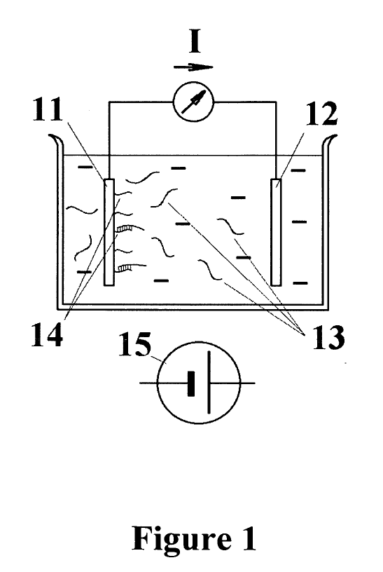

Embodiments of the present invention will be described below, referring to the drawings. FIG. 1 is a schematic representation of an electrode assembly of a single cell unit, presented herein for better understanding of operational principles of the integrated circuit array sensor. The cell in FIG. 1 is comprised of a sensitive electrode 11; a counter electrode 12; a sample substance containment into which the electrodes are disposed and into which target molecular structures for analysis 13 may be introduced; and means for detecting voltage and electric current across the working electrode—counter electrode pair. To detect the presence of a particular type of target molecular structures in a sample substance, the sensitive electrode 11 could be modified by immobilizing the probe 14, which is capable of bonding 15 with an associated target molecular structure. Symbolic representation of a cell unit having the functionality of said electrode assembly is given by 16.

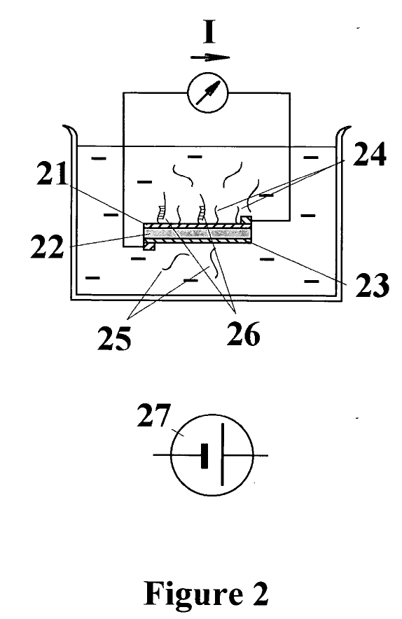

In yet another embodiment of this invention, FIG. 2 is a schematic representation of an electrode assembly of a single cell unit, comprising an ultra-thin metal film 21 deposited on a surface of semiconductor material 22, which said semiconductor material has specific conductive properties, and counter electrode 23 which is arranged such that the components 21, 22, and 23 have a functionality of metal-semiconductor diode detector. To detect the presence of a particular type of target molecular structures in a sample substance, the sensitive electrode 21 could be modified by immobilizing the probe 24, which is capable of bonding 26 with an associated target molecular structure 25. Symbolic representation of a cell unit having the functionality of said electrode assembly 21-23 is given by 27.

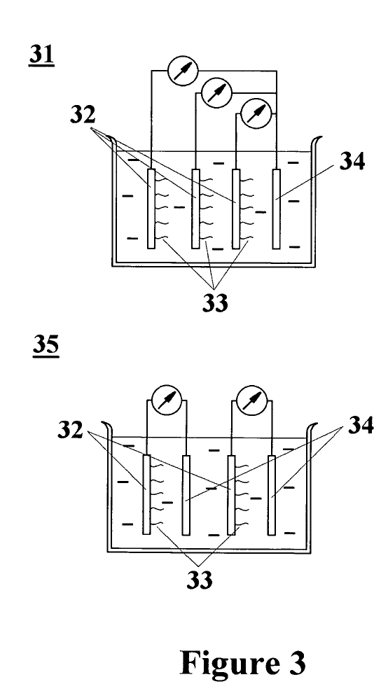

FIG. 3 is a schematic representation of a multi-electrode assembly, comprising a plurality of sensitive electrodes 32, where each sensitive electrode could be modified by immobilizing a particular type of probe of molecular structure 33. Each said probe is capable of bonding with an associated target molecular structure. Therefore, the multi-electrode assembly is capable of identifying the presence (or absence) of a plurality of target molecular structures in parallel. Said multi-electrode assembly can be implemented either with a single counter-electrode common to all sensitive electrodes of the assembly, as can be seen in FIG. 3-31, or, in another embodiment of this invention illustrated in FIG. 3-35, said multi-electrode assembly can be implemented as a plurality of pairs of sensitive electrodes and corresponding counter-electrodes, similar to the assembly shown in FIG. 1, or equally acceptable, similar to the assembly shown in FIG. 2. Here each said pair of electrodes is capable of bonding with a particular type of target structure in a sample substance.

A single cell unit in FIG. 1 resembles a cell of an electrochemical battery in that both can produce a voltage and flow of electric current in external circuitry. In the present invention, however, an electromotive force of a different nature can be exploited. Some examples include, but are not limited to, (a) an oxidation/reduction on the surface of the sensitive electrode due to forming or breaking duplexes of probe-target molecular structures; (b) producing voltage and current due to a gradient of concentration of electrolytic ions in a sample substance created due to the reaction of probe and target molecular structures, see V. Bagotzky, “Fundamentals of Electrochemistry”, 107-112 (Plenum Press, New York-London, 1973); (c) producing voltage and current due to low energy chemisorption reactions on the surface of the sensitive electrode of the metal-semiconductor diode detector, schematically presented in FIG. 2, through the use of mechanisms known from prior art, see Gergen et al, “Chemically Induced Electronic Excitation at Metal Surfaces”, Science 294, 2521-2523 (2001).

However, the voltage and current produced by a single cell unit schematically shown in FIG. 1 and FIG. 2 often might be too low to be detected by conventional means for electrical measurements, such as a voltmeter or an ammeter. To achieve the level of sensitivity required for reliable detection of the electrical signal produced by an interaction of probe and target molecular structure, the present invention provides an integrated circuit array sensor comprised of a set of miniatured pixel units. Each said pixel comprises a single cell unit and associated access circuitry. Each said pixel is usually not bigger than (1 mm×1 mm) in size, and most preferably is less than (100 m×100 μm) in size. Herein the benefits of reducing the size of the pixel for increasing sensitivity can be linked to lower capacitive current, smaller ohmic drop and faster achievement of mass transport in a stationary diffusion state on the sensitive electrode of the cell unit.

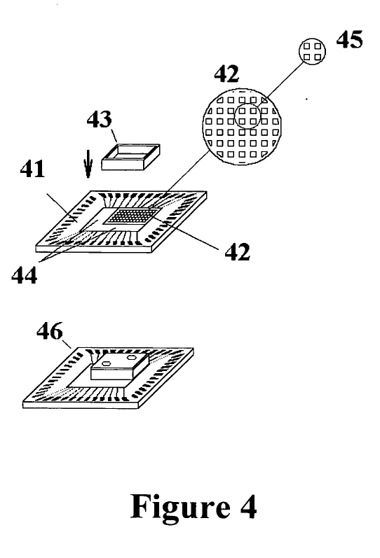

FIG. 4 is a schematic representation of an assembly of a single integrated array sensor 41, comprising a set of sensitive pixels 42, and a hybridization chamber 43. Each said sensitive pixel of the array 42 is capable of accumulating electrical charge upon interaction of probe and target molecular structures. The hybridization chamber 43 is installed on the sensitive area of the sensor such that filling said chamber with a sample substance thereby exposes an array of pixels to that substance. Optionally, additional amplifiers and data access circuitry 44 can be allocated on the substrate, upon which an array of sensitive pixels is manufactured. Insert 45 in FIG. 4 shows the arrangement of individual pixels in the array of said array sensor whereby 46 presents an assembly of the sensor and hybridization chamber.

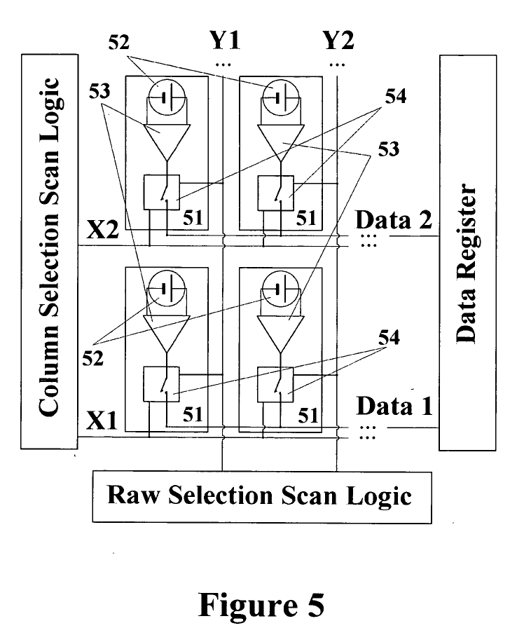

To illustrate one embodiment of this invention, FIG. 5 shows a 2×2 fragment of the array sensor with individually addressable active pixels, which bear a resemblance to the technology and design known for CMOS array sensors, see U.S. Pat. No. 6,344,877 and references therein. However, in this invention photosensitive elements are replaced by a cell unit 52, where each said cell unit has the functionality of the cell units shown and described in detail in FIG. 1, or equally acceptable, shown and described in FIG. 2. Each sensitive pixel also comprises optional amplifying circuitry 53 and X-Y electronically addressable switch 54 for outputting a pixel signal in response to a request from the data access circuitry.

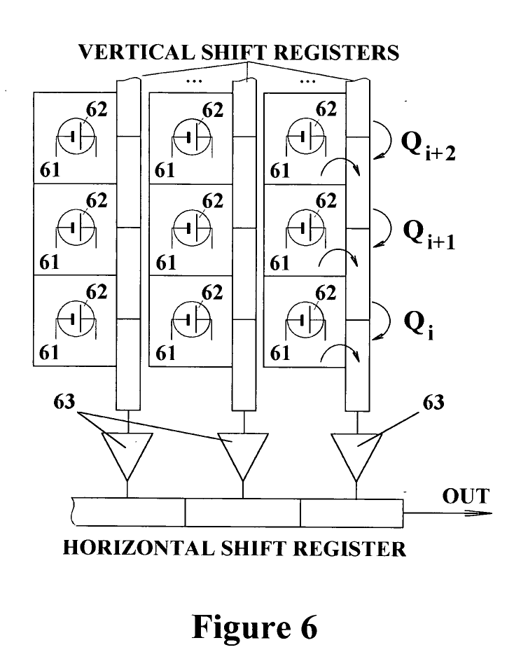

In yet another embodiment of this invention, FIG. 6 shows a schematic representation of a 3×3 fragment of the array sensor comprised of pixel units 61. The sensor herein is implemented using technology known from prior art for CCD array imagers. According to the present embodiment, corresponding pixel units of a CCD sensor comprises cell units 62, where each cell unit 62 has the functionality and design similar to the cell unit described hereinabove in FIG. 1, or equally acceptable, similar to the cell unit described in FIG. 2. In FIG. 6, the output electronic signal representative of the electric charge, Qᵢ, accumulated by a i-th pixel first is transferred to a storage cell of a corresponding vertical shift register, then is sequentially transferred to the horizontal shift register where the charge can be amplified and outputted to the external data processing circuitry. The number of pixels in the array sensor is not limited to any particular number of pixels, can exceed hundreds of pixels, and most preferably exceeds thousands of pixels.

The present invention also provides a method for identifying molecular structures within a sample substance, comprising the steps of:

(a) Applying the substance to a plurality of test sites formed on a surface of said integrated circuit array sensor, said test sites having respective probes attached thereto which specifically bind to a target molecular structure (hereinafter “target”), such that different test sites have probes which specifically bind to different targets; and such that each test site covers at least one pixel circuitry of said array device.

In operation, the test sites, described in greater detail herein below, are formed on said sensor array area using standard techniques of immobilization of biopolymer molecules on metal or semiconductor substrate. Each test site contains a plurality of probes, which are capable of binding to known molecular structures. The targets could comprise, for example, biopolymers, such as polynucleotides, DNA, RNA, and their known analogs, proteins, peptides, ligands or antibodies. Different probes are used in test sites for simultaneous detection of a plurality of different targets within a sample substance.

The probes are attached to the test sites by fixation to the sensitive electrodes of the array sensor. The sensitive electrodes must be treated and functionalized to create a surface chemistry conducive to the formation of covalent linkages with the selected probes. As an example of oligonucleotide immobilization, the sensitive electrodes can be functionalized with an epoxide group by reaction with an epoxy silane. The epoxide group on the support reacts with a 5′-amino-derivatized oligonucleotide probe to form a secondary amine linkage, as described in Parham and Loudon, “Carboxyl-Terminal Sequential Degradation of Peptides”, Biochem. Biophys. Research Comm 1, 1-6 (1978), which is incorporated by reference herein. Formation of this covalent linkage attaches the probes to the support surface of the sensitive electrode. Another example of functionalization of the polymer-treated surfaces include 5′-aldehyde or carboxylic acid derivatives coupled to hydrazide-activated polystyrene as described in Kremsky, et al, “Immobilization of DNA via oligonucleotides containing an aldehyde or carboxylic acid group at the 5′ terminus”, Nucl. Acids Res. 15, 2891-2909, (1987), and 5′-amino derivatives coupled to polystyrene which has been activated by diazotization and 5′-phosphate derivatives coupled to amino-functionalized polystyrene as describe in Lund, et al, “Assesment of method for covalent binding of nucleic acids to magnetic beads, Dynabeads, and the charcteristics of the bound nucleic acids hybridization reaction”, Nucl, Acids Res. 16, 10861-10880, (1988), both articles being incorporated by reference herein.

For direct attachment of probes to the sensitive electrodes of the array sensor, the surface of electrodes must be fabricated with materials capable of forming conjugates with the probes. Materials which can be incorporated into the surface of the plates to provide for direct attachment of probes include metal materials such as gold, silver, aluminum, copper, iridium, platinum, titanium, and semiconductor materials such as silicon, germanium, and semiconductor composites known in the art. These metals are capable of forming stable conjugates directly on the plate surface by linkages with organic thiol groups incorporated into the probe as described in Hickman, et al, “Selective Functionalization of Gold Microstructures with Ferrocenyl Derivatives via Reaction with Thiols or Disulfides: Characterization by Electrochemistry and Auger Electron Spectroscopy”, J. Am. Chem. Soc. 113, 1128-1132 (1991), which is incorporated by reference herein. As an example, a synthetic DNA probe labeled with a thiol group at either the 5′ or 3′ terminus will form a stable conjugate with a metal such as gold in the sensor surface to create an array of directly attached probes. To form conjugates with semiconductor materials, such as silicon, the surface first can be modified by immobilizing a linker group to which amino-modified probes thereby can be attached, see Yamada et al, “Application of Organic Monolayers Formed on Si(111): Possibilities for Nanometer-Scale Patterning”, Electrochem. Commun., 3, 67 (2001), which is incorporated by reference herein.

(b) Maintaining a constant preprogrammed temperature of the substance and said array sensor, or alternatively, running a preprogrammed temperature profile such as, but not limited to, gradually increasing or decreasing the temperature or a stepwise change of the temperature.

Temperature is an important factor that affects the rate of probe-target interaction. Raising the temperature above a specific value can inhibit the binding reaction, or in some other cases can reverse the pathway of the reaction causing breakage (melting) of already formed probe-target duplexes on the sensor surface. Therefore, maintaining a specific temperature profile can provide additional means for identifying targets. For example, target molecular structures can be identified using the specific value of temperature at which the reaction, such as melting or breaking bonds, occurs in response to a gradual increase or decrease of the temperature of the sample substance. Alternatively, target molecular structures can be identified based on the rate of forming or breaking bonds with molecular structures of the associated probe site in response to a stepwise change of temperature. Furthermore, a stepwise change of temperature provides the additional benefit of increasing detection sensitivity, increasing the magnitude of electronic signals from probe sites by forcing the reaction of melting or breaking bonds that occur within a short period of time driven by the rise of the temperature above the melting point.

To maintain a desirable temperature profile, the sensor and associated components of the apparatus can be placed either into an oven such as a hybridization oven known from the art; or by using a thermocycler commonly used for running pre-programmed temperature profiles; or by using other means for maintaining temperature profiles known from prior art.

(c) Acquiring an electronic signal from a plurality of the pixels associated with the test sites, each test site covering at least one pixel of said array sensor.

To acquire an electronic signal from pixels of the array sensor, a controller unit and an input/output port for communicating with external control, post-processing, and data storage circuits, such as a computer, can be provided similar to those known in the art, for example, for interfacing CMOS and CCD array imagers. The output data can be further processed using means and algorithms for signal processing for the purpose of reducing noise and extracting quantitative characteristics of the signal associated with specific test sites of the array sensor.

(d) Detecting the amplitude of the electronic signal versus time from the test sites to determine which probes have interacted with an associated target molecular structure such that a plurality of different targets can be detected.

### EXAMPLES

The functions and advantages of the present invention will be more fully understood from the examples below. The following examples are intended to illustrate the benefits of the present invention, but do not exemplify the full scope of the invention. References cited in the following examples are incorporated herein by reference for all purposes.

### Example 1

### Benefits of Miniaturization Pixel Units

Introduction of a new type of electronically sensitive array sensor for detection and identification of molecular structures can address issues of both increasing sensitivity and performing analysis of many sampling spots in parallel. The sensitivity of detection of electrical current by a conventional bioelectronic sensor, in which the electrode and amplifying electronics are separated, is often limited to 10-100 pA. The detection sensitivity can be increased if the sensitive electrode and amplifying electronics are miniaturized and manufactured on a single sensor substrate, similar to what is known in the art for optical image array sensors, see Kempainen, “CMOS Image Sensors”, EDN Access (October, 1997); Suni, “Custom Photodetector Arrays Meet Design Challenges”, Laser Focus World (1994); and Suni, 7^{th} Annual IEEE International Conference on Wafer-Scale Integration (1995), all three articles being incorporated by reference herein. Indeed, video CMOS arrays are capable of detecting signals with a noise level equal to 1000 electrons/pixels acquired over a 20-100 msec time interval. This corresponds to “electrical dark current” (noise) of(1000×1.6×10⁻¹⁹ C/20 msec)=0.008 pA. The current is detected from a single pixel typically having an area of about 20×20 μm or so. By recalculating the current for an area of CMOS element of 100×100 μm (5×5 pixels), one finds the magnitude of the noise of CMOS-based sensor is (25^{1/2}×0.008 pA)=0.04 pA, versus 10-100 pA for a conventional bioelectronic sensor with a comparable size. The dark current or noise of CCD imagers typically is one order of magnitude lower than the noise of CMOS sensors, therefore promising even better detection sensitivity than the estimates hereinabove for CMOS-based technology.

In addition, current technologies for manufacturing CMOS and CCD electronic chips allow hundreds of thousands of sensitive pixels to be produced on a single silicon substrate, and so allows a plurality of test sites to be used for simultaneous detection of multiple targets in a sample substance.

### Example 2

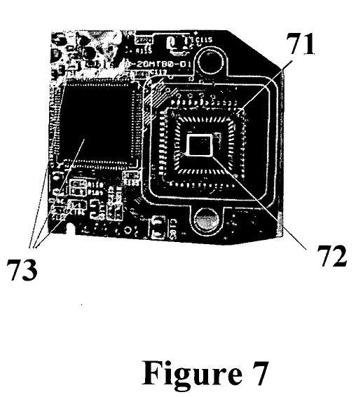

FIG. 7 shows a prototype of an apparatus that was used in preliminary studies of the integrated array sensor for detection of biomolecules in solution. In FIG. 7, the array sensor 71 is shown with a hybridization chamber 72 set up on the sensitive area of the sensor. The array sensor was interfaced with commercially available “camera-on-board” circuitry 73 capable of digitizing and transferring data to a personal computer where the data can be further processed, visualized and stored. Arrangement of the sensitive pixels of the sensor in a 2D array allows for the capture, processing and presentation of data the way it is known in the art for image sensing array devices.

### Example 3

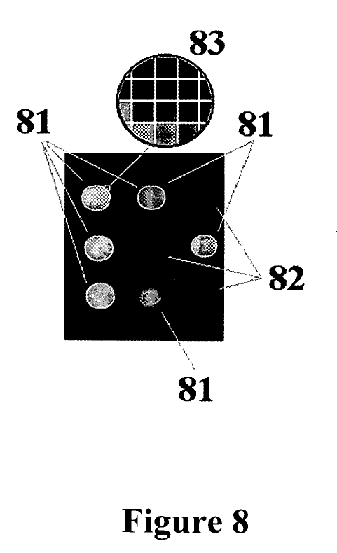

A commercially available CMOS array sensor for imaging applications was modified by 5′-end thiohexyl modified oligonucleotides, immobilized on the sensor's surface as an array of 3×3 test sites. A set of two probes and one target oligo were used in these studies. The oligos were custom synthesized by AlphaDNA (Montreal QC, Canada). The probe oligos were 5′-end thiohexyl modified for immobilization on metal interconnects on the surface of the array sensor. One probe oligo, Probe-1, had the sequence 5′-Thiol-ggaat aaaat tgatt cattt taaaa aaaaa-3′, which is homologous to a 30-base long fragment of Interleukin-6, Locus HSU62962 (7p21). The other probe oligo, Probe-2, had the sequence 5′-Thiol-cctgg ccacc gcgtg ctgct gctgc ggggg-3′, herein representing a 30-base long fragment from the Locus CACNL1A4 (19p13). The presence of a sulfide group at the end of the thiol-modified oligo enables the attachment of the oligo molecule to a metal component and interconnects on the sensitive area of the chip. In the previous art thiol-modified oligos were used for colorimetric differentiation of polynucleotides and in high sensitivity scanometric assay for hybridization detection, see Taton, et al, “Scanometric DNA Array Detection with Nanoparticle Probe”, Science, 289, 1757-1760 (2000). In our preliminary studies, the test spots on the sensitive area of the chip were produced by 1 μl of 100 pM solution of corresponding thoxehyl-modified probe oligo placed directly on the sensitive area of the array sensor. To bind oligos to the surface, the sensor was incubated at room temperature for one hour in a humidified chamber, which prevents spots of the test array from drying. The sensor surface then was washed with a TE buffer (Tris-EDTA buffer, Research Organics, Cleveland, Ohio) and rinsed twice with de-ionized distilled water. A sample substance, containing target 30-base long non-modified oligos with a sequence homologous to the Probe-1 was prepared in a hybridization buffer following a protocol from the known art, see Morel and Cavalier, “in situ Hybridization in Light Microscopy”, p.130, CRC Press, Boca Raton, Fla. (2000), which is incorporated herein by reference. The hybridization chamber 73 of the array sensor shown in FIG. 7 was filled with 50 μL of 100 pM solution of the target oligo. Hybridization reaction was pursued at 30° C. To protect the acquiring signal from ambient light, the apparatus was enclosed in an opaque container. FIG. 8 shows the 2D pattern acquired from the sensor as the hybridization proceeds. In FIG. 8, the test spots 81 carrying oligos homologous to the target exhibit a superior electronic signal versus the test spots 82, in which the Probe-2 oligo is non-homologous to the target Insert 83 shows the signal pattern from individual pixels of the array sensor. The experimental results hereinabove illustrate the concept and procedures of preparing a sensor for the analysis of target molecular structures in a sample substance.

### Example 4

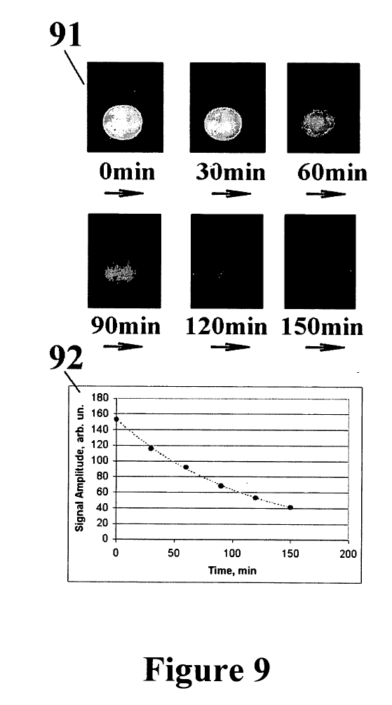

FIG. 9 shows the electronic signal versus time acquired from a single probe site where hybridization proceeds as described hereinabove in Example 3. A set of data similar to that shown in FIG. 9 can be used to find the rate of hybridization corresponding to each particular probe site. The measurements versus time herein provide an additional means for identifying the target molecular structure based on quantitative characteristics of the hybridization process. Plot 91 in FIG. 9 shows the amplitude of an electronic signal versus time measured by averaging the signal from corresponding pixels of the test site. The rate of reaction in this particular example is determined by fitting the experimental data versus the exponential dependence of the signal versus time given by A˜exp(-kt), where A is the amplitude of the electronic signal, k is the rate of reaction, and t is the time. When applied to DNA in solution, hybridization follows the kinetics of a second order reaction. The hybridization rate constant, k₂, decreases with increasing strand length: k₂˜L⁻⁰⁵, where L is the number of nucleotides per strand, see Wetmur and Davidson, “Kinetics of Renaturation of DNA”, J. Mol. Biol., 31, 349-370 (1968). Hybridization of oligonucleotides on a solid support can be approximated by the kinetics of a one-step adsorption reaction, see Spigelman, J. G Wetmur, “Acceleration of DNA renaturation rates”, Biopolymers, 14, 2517-2524 (1975). Similar to the reaction in solution, the rate of hybridization reaction on a surface, k, is exhibiting dependence versus the number of overlapping bases in the probe and target strands according to k˜L^{˜05}, where L is the number of overlapped bases in the strands. Therefore, the quantitative measurement of the reaction rate can be used, for example, for distinguishing perfect and non-perfect matches of probe and target sequence based on the specific values of the reaction rate, k, where k˜L^{-0.5} for a perfect match and k˜(L-1)^{-0.5} for a single base mismatch of the probe and target sequences.

### Example 5

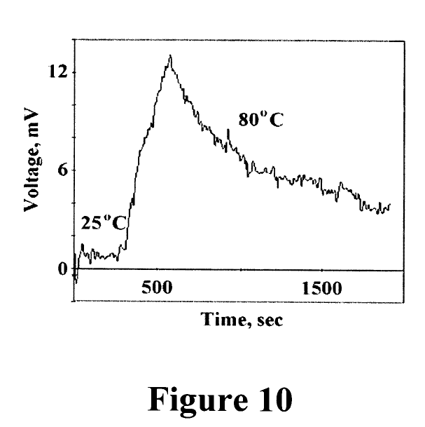

The experimental setup illustrated in FIG. 1 was used to study the electronic response upon denaturing (melting) oligonucleotides. The synthetic thiohexyl-modified probe oligo, Probe-1, described hereinabove in Example 3, was immobilized on a surface of 0.1 mm×4 mm gold electrode. The oligo was immobilized by incubating the electrode in 100 pmol solution of the Probe-1 oligo for one hour at room temperature. After washing and rinsing, said modified electrode and non-modified gold counter electrode were placed in a micro-tube, thereby forming a cell similar to that in FIG. 1. For temperature control, the microtube with electrodes was placed in a thermocycler (Cetus, Perkin Elmer). The thermocycler allows the programmed running of different temperature profiles, including stepwise changes in temperature occurring in less than 15 sec steps, or gradual, smooth changes in temperature over a predefined time interval. The tube was filled with a solution of the target oligo with a sequence perfectly complementary to the probe oligos. Modified probe electrode and target oligos were hybridized overnight at room temperature. The tube then was placed in the thermocycler, and voltage between the probe (modified) and reference (non-modified) electrodes was recorded versus time as the temperature was raised from initial room temperature to 80° C., which is well above the melting temperature of the probe-target duplexes. FIG. 10 shows the voltage between electrodes versus time, which was acquired by raising the temperature stepwise at t=300s from room temperature to the final temperature of 80° C. In the figure, the voltage first sharply increases and reaches its maximum value at t=550s, then gradually decreases as new equilibrium conditions are established. The maximum value of the signal observed upon melting of the probe-target duplexes was significantly higher than the magnitude of electronic response upon hybridization, which in this experiment was barely distinguishable from the electronic noise of the detection system. The gain of the signal upon denaturation can be understood by considering the fact that breaking duplexes upon denaturation happened “all-at-once” during a relatively short period of time of about 15 min as can be found from the data set in FIG. 10. The formation of hybridized duplexes is a much slower process that is often controlled by diffusion and requires hours to complete. The signal detected in this experiment is proportional to the electric current, the faster process causing the stronger signal. Therefore, in this example the stepwise change of temperature provides an additional benefit by increasing sensitivity when detecting the interaction of probe and target molecular structures.

### Example 6

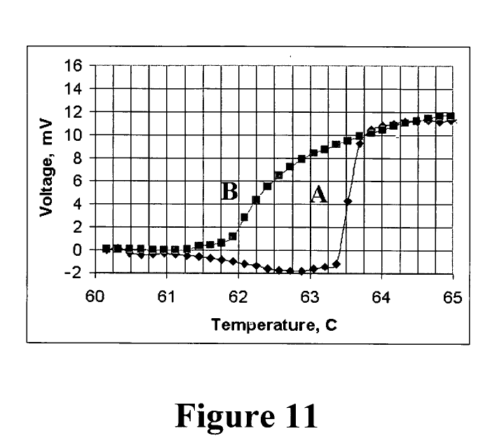

FIG. 11 shows two sets of data obtained by denaturing hybridized probe-target complexes using the experimental setup and procedures described in Example 5, in case (A) when the target is perfectly complementary to the probe; and case (B) when one G base in the target sequence is replaced by T (point mutation). In this experiment, the temperature of the solution was raised from room temperature at a constant rate of 2.75° C./min. No response was observed before the temperature reached 61° C. Then the voltage between electrodes rose sharply to the maximum value, similar to what was observed in the previous experiment shown in FIG. 10. In FIG. 11, the amplitude of the signal is plotted versus temperature of the sample solution. One can notice in FIG. 11 that the oligos with mutation (B) melted at a temperature of 1.3° C. lower and exhibited broader melting transition than the perfectly complimentary probe-target duplexes (A). The result is consistent with what can be expected for interaction of perfectly and non-perfectly matching DNA. These preliminary results indicate the proposed technique can be used for Single Nucleotide Polymorphism (SNP) analysis while most other hybridization methods have difficulties with reliable SNP determination.

1. An apparatus for identifying molecular structures within a sample substance, comprising:
an integrated circuit array sensor for outputting signals representative of the interaction of probe molecular structures on the surface of said sensor and target molecular structures within a sample substance (hereinafter “target”);
a controller unit for interfacing with said integrated circuit array sensor and accessing said signals representative of the interaction of probe and target molecular structures;
an input/output port for communicating with control, post-processing, and data storage circuit;
a chamber, such as a hybridization chamber, for exposing sensitive area of said integrated array sensor to the sample substance; and
a means for controlling and programmable changing of the temperature of said chamber, the sample substance and said integrated array sensor.

2. The integrated array sensor of claim 2, where said sensor comprises a set of pixel circuits, each said pixel is usually not bigger than (1 mm×1 mm) in size, and most preferably is less than (100 μm×100 μm) in size;
each said pixel is capable of accumulating and processing an electric charge produced upon interaction of probe and target molecular structures on the surface of said sensor.

3. The integrated array sensor of claim 1, where said sensor comprises an array of active pixels, each said pixel has one or more active transistors within the pixel unit, and each said pixel has a connection with a multiple column readout circuit, similar to the architecture implemented by Complementary Metal Oxide Semiconductor (CMOS) array imagers. Said active pixels are capable of converting the electric charge accumulated by said pixels into an output electronic signal.

4. Alternatively, the integrated array sensor of claim 1, where said sensor comprises an array of pixels capable of accumulating, storing, and transferring an electric charge to a readout register formed in the sensor's substrate, similar to the design implemented by a Charge-Coupled Device )CCD). Said pixels and readout circuits are capable of converting an electric charge accumulated by said pixels into an output electronic signal.

5. The chamber of claim 1, where said chamber is attached thereto to the sensitive area of said integrated array sensor such that filling said chamber with a sample substance exposes the sensitive area of the integrated array sensor to that sample substance.

6. The means for controlling and changing temperature of claim 1, where said means allows maintaining a constant preprogrammed temperature of the sample substance and said integrated array sensor, or alternatively, said means allows running a preprogrammed change of temperature versus time such as, for example, gradually increasing or decreasing the temperature, or a stepwise change of temperature. Said means for controlling and changing temperature also are capable of providing an electronic output versus time representative of the temperature of the sample substance and the integrated array sensor.

7. (canceled)

8. (canceled)

9. The method of claim 7 wherein said probes are antibody probes.

10. The method of claim 7 wherein said probes are ligands or ligand analogs capable of binding to proteins.

11. The method of claim 7 wherein said probes are protein or peptide probes.

12-16. (canceled)

17. A method for identifying molecular structures within a sample substance, comprising the steps of:
(a) applying the substance to a plurality of test sites formed on a surface of an integrated circuit array sensor, the integrated array sensor operable to detect electrical signals produced on the test sites in the absence of any external application of external electrical signals to the test sites, the test sites having respective probes attached thereto which specifically bind to a target molecular structure, such that different test sites have probes which specifically bind to different target molecular structures;
(b) controlling the temperature of the substance and said integrated circuit array sensor;
(c) acquiring an electronic signal from a plurality of the pixels associated with the test sites, each test site covering at least one pixel of said integrated circuit array sensor; and
(d) detecting, in the absence of any external application of external electrical signals to the test sites, the amplitude of the electronic signals produced on the test sites versus time to determine which probes have interacted with an associated target molecular structure such that a plurality of different targets can be detected.

18. The method of claim 17, wherein the step of controlling the temperature of the substance and said integrated circuit array sensor comprises maintaining a constant preprogrammed temperature of the substance and said integrated circuit array sensor.

19. The method of claim 17, wherein the step of controlling the temperature of the substance and said integrated circuit array sensor comprises increasing or decreasing the temperature of the substance and the integrated circuit array sensor as a function of time.

20. The method of claim 17, wherein the step of controlling the temperature of the substance and said integrated circuit array sensor comprises stepwise changing of the temperature of the sample substance and said integrated circuit array sensor.
